(19) United States
(12) Patent Application Publication
(10) Pub. No.: US 2008/0085246 A1
(43) Pub. Date: Apr. 10, 2008

(54) Use of Eugenol Acetate as an Agent Against Bad Breath

(75) Inventors: Jürgen RABENHORST, Höxter (DE); Arnold MACHINEK, Holzminden (DE); Steffen SONNENBERG, Markkleeberg (DE); Gerald REINDERS, Höxter (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/575,905

(22) PCT Filed: Sep. 20, 2005
(86) PCT No.: PCT/EP05/54705

§ 371 Date: Mar. 23, 2007

(30) Foreign Application Priority Data
Mar. 29, 2004 (FR) 0403207

(51) Int. Cl. A61K 8/37

(52) U.S. Cl. 424/49

(57) ABSTRACT

The use of eugenol acetate as an agent (i) for inhibiting and/or preventing the growth of and/or for killing microorganisms which cause bad breath and/or (ii) for combating bad breath is described.

The invention relates to the use of eugenol acetate as an agent against bad breath and corresponding methods. The invention furthermore relates to oral hygiene products comprising eugenol acetate.

The healthy human mucous membrane of the oral and pharyngeal cavity and the solid tooth substance are colonized by a large number of non-pathogenic micro-organisms. This so-called microflora of the oral cavity is not only harmless, it represents important protection for combating opportunistic or pathogenic germs.

One considerable problem of oral hygiene is offensive breath, also called bad breath, foetor ex oris or halitosis. This odour is formed by decomposition of residues of food and dead cells of the mucous membrane by microorganisms. The attack by Gram-positive and Gram-negative bacteria causes the bad breath. Anaerobic Gram-negative bacteria are mentioned above all as causative organisms in the literature (e.g. Bad Breath—A Multidisciplinary Approach. eds: D. van Steenberghe, M. Rosenberg, Leuven University Press, Leuven 1996; 111-121).

Since social contacts are often impeded by bad breath, those affected have a great interest in remedying or preventing it.

Bad breath is to be differentiated here from pathological ulcerative stomatitis (stomatitis ulcerosa). While bad breath is a normal characteristic of human breath and therefore a merely cosmetic problem, ulcerative stomatitis is a disease. It is accompanied by a characteristic, very unpleasantly smelling note in the breath.

The object of the present invention was to discover active substances and agents against bad breath or against the microorganisms involved in the formation thereof.

Eugenol acetate (4-allyl-2-methoxyphenyl acetate; CAS No. 93-28-7) is known per se and can be represented by the following formula:

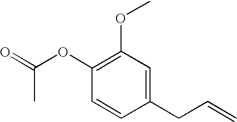

The smell of eugenol acetate can be described as clove-like, sweet and balsamic-fruity.

Clove oils are obtained from the flower buds, stem or leaves of the clove plant, each of these containing different amounts of the main components eugenol, eugenol acetate and caryophyllene.

The main constituent of clove oils is eugenol, which is responsible for the smell thereof and above all for the antimicrobial, antiseptic and analgesic action of clove oils. Eugenol has an antimicrobial activity against a large number of Gram-positive and Gram-negative bacteria.

The clove oils with the highest content of eugenol acetate are the oils which are obtained from the flower buds and which typically contain 75-90 wt. % eugenol, 4-15 wt. % eugenol acetate and 5-12 wt. % beta-caryophyllene. The weight ratio of eugenol acetate to eugenol in clove oils here is less than 1:5.

Clove oils are in some cases employed in undiluted form in dental medicine.

In the field of oral care compositions, dilute clove oils, conventionally 1-2% strength solutions, can be used as a mouth wash.

DE 19612340 (corresponds to U.S. Pat. No. 6,514,551) describes a process for improving the life of and/or stabilizing microbially perishable products by means of a process auxiliary which comprises at least one microbicidally acting aroma substance, eugenol acetate also being mentioned among a large number of aroma substances. This process auxiliary can be employed, for example, in the form of lubricants, emulsifying and cleaning compositions and cutting or release agents. In this procedure, only the surfaces or cut areas of the foodstuffs are charged with the process auxiliaries. The activity against various microorganisms, in particular organisms which cause decay (e.g. A. niger, Enterobacter, Lactobacilli) but which play no role in the formation of bad breath, has been investigated.

The present invention primarily relates to the use of eugenol acetate as an agent (i) for inhibiting and/or preventing the growth of and/or for killing microorganisms which cause bad breath and/or (ii) for combating bad breath.

The present invention furthermore relates to a method of inhibiting and/or preventing the growth of and/or for killing microorganisms which cause bad breath with the following step:
bringing microorganisms which cause bad breath into contact with an amount of eugenol acetate which is antimicrobially active against these microorganisms.

The invention also relates to a method of combating bad breath with the following step:
introduction of an amount of eugenol acetate which is antimicrobially active against microorganisms which cause bad breath into the oral cavity and/or the pharyngeal cavity.

The invention also relates to an oral hygiene product containing eugenol acetate in an amount of at least 0.01 wt. %, based on the total weight of the oral hygiene product, wherein the weight ratio of eugenol acetate to eugenol is greater than 1:5 if the oral hygiene product also contains eugenol.

Further advantageous embodiments of the invention emerge from the patent claims, the following description and the examples.

In the present text, oral hygiene products are understood as meaning the formulations familiar to the person skilled in the art for cleansing and care of the oral cavity and the pharyngeal cavity and for refreshing the breath. Known and customary oral hygiene formulations are both creams, gels, pastes, foams, emulsions, suspensions, aerosols and sprays, and capsules, granules, pastilles, tablets, bonbons or chewing gums, without this list of presentation forms being limiting in respect of the possible uses. Such formulations serve to cleanse and care for the tooth substance and oral cavity and to refresh the breath.

The eugenol acetate to be employed according to the invention is a compound which can be incorporated largely universally into the most diverse presentation forms of oral hygiene products without having to be limited to one or a few specific presentation forms, i.e. eugenol acetate harmonizes with a very large number of conventional cosmetic auxiliary substances and additives.

It has furthermore emerged that eugenol acetate can both effectively reduce or eliminate bad breath, and prevent formation thereof, i.e. is also capable of acting preventively.

It has furthermore emerged that the oral hygiene products according to the invention are active over a relatively long period of time, and in particular of the order of at least three hours, without their action decreasing noticeably.

Eugenol acetate effectively combats bad breath without noticeably harming the physiological flora of the oral and pharyngeal cavity.

The prior art has provided no indication of the use according to the invention of eugenol acetate as an agent for reducing or eliminating bad breath or preventing the formation of bad breath.

It has been found that eugenol acetate can completely or partly prevent the growth of Gram-positive and Gram-negative bacteria of the oral and pharyngeal cavity which are responsible for bad breath.

It has been found that eugenol acetate can completely or partly prevent the formation of components which cause bad breath.

In particular, eugenol acetate is capable of inhibiting and/or preventing the growth of microorganisms which cause bad breath which are chosen from the group consisting of: Eubacterium, Fusobacterium, Haemophilus, Neisseria, Porphyromonas, Prevotella, Treponema and Veillonella species, in particular Fusobacterium nucleatum, Porphyromonas endodontalis, Porphyromonas gingivalis, Prevotella intermedia, Prevotella loeschii and Treponema denticola.

Eugenol acetate is distinguished in particular by a very good action against anaerobic and microaerophilic bacteria.

It was furthermore astonishing that eugenol acetate has a particularly good action against the particularly pronounced morning bad breath which is typically to be perceived in the morning after getting up.

In addition, it has emerged that eugenol acetate can completely or partly prevent the decay of oral hygiene products due to attack by Gram-positive and/or Gram-negative bacteria when it is added to these products in an active amount. Eugenol acetate can thus also simultaneously act as a preservative here.

An oral hygiene product having a content of eugenol acetate in the range of from 0.01 (100 ppm) to 10 wt. %, in particular having a content of from 0.025 (250 ppm) to 5 wt. %, preferably having a content of from 0.05 (500 ppm) to 3 wt. %, and particularly preferably 0.08 (800 ppm) to 3 wt. %, based on the total weight of the product, is preferred according to the invention.

In a very particularly preferred embodiment, the content of eugenol acetate is in the range of from 0.1 to 2 wt. %, based on the total weight of the product.

If oral hygiene products according to the invention containing eugenol acetate also contain eugenol, a weight ratio of eugenol acetate to eugenol of greater than 1:3 is advantageous, and a weight ratio of greater than 1:1 is preferred, particularly preferably greater than 2:1 and very particularly preferably greater than 10:1.

Oral hygiene products which contain only a very small amount of eugenol or no eugenol are preferred according to the invention. In a preferred embodiment, the oral hygiene products according to the invention are therefore substantially free from eugenol. In the present case, substantially free from eugenol means that the oral hygiene product contains eugenol at most in an amount which is not active against bad breath.

Eugenol acetate-containing oral hygiene products which contain the eugenol acetate not in the form, i.e. not as a constituent, of a clove oil are preferred.

Disadvantages of eugenol are the sensory profile thereof and discoloration problems which are known for phenolic compounds in some formulations, media or carriers. The taste of eugenol is very clove-like and phenolic, and is often found to be unpleasant and medicinal, including in oral hygiene products.

The taste of eugenol acetate (in oral hygiene products), especially at relatively high doses, also is not always found to be pleasant, although this is significantly less clove-like and medicinal than that of eugenol.

In the context of the present invention, there was thus the subsequent object of achieving a modification of the taste of the oral hygiene products according to the invention containing eugenol acetate such that these have an improved, pleasanter and less clove-like and/or medicinal taste.

It has been possible to achieve this subsequent object by an oral hygiene product according to the invention containing an aroma composition comprising menthol. In this context, menthol not only imparts the menthol-specific cooling freshness, but at the same time reduces the clove-like and medicinal taste impression of the eugenol acetate.

A further aspect of the present invention relates to an oral hygiene product containing 0.01 (100 ppm) to 10 wt. %, preferably 0.05 to 3 wt. % eugenol acetate and an aroma composition containing menthol.

An aroma composition in the context of the present invention comprises menthol and one or more further substances having a sensory action.

In this context, a weight ratio of eugenol acetate to aroma composition in the range of 50:1 to 1:50, preferably from 20:1 to 1:20 is preferred.

The weight ratio of eugenol acetate to menthol, i.e. the sum of all the menthol isomers, is advantageously in the range of 10:1 to 1:10, preferably in the range of 5:1 to 1:5. Of the menthol isomers, racemic menthol and I-menthol are preferred.

Menthol can be employed here in the pure form (natural or synthetic) and/or as a constituent of natural oils and/or menthol-containing fractions of natural oils, in particular in the form of peppermint oils, such as Mentha arvensis or Mentha piperita or menthol-containing fractions thereof.

It is of course possible and advantageous to prepare a mixture of eugenol acetate and an aroma composition comprising menthol, to subsequently incorporate this into an oral hygiene formulation and to prepare an oral hygiene product according to the invention in this manner.

It has moreover been found that an oral hygiene product according to the invention has a taste which is improved further if it comprises an aroma composition which also contains, in addition to menthol, one or more substances having an aniseed note.

Advantageous substances having an aniseed note are: anethole, anisole, aniseed oil, star aniseed oil and fennel oil, and in addition also anisaldehyde and anisyl alcohol. Anethole and anethole-containing essential oils are preferred. Substances having an aniseed note, in particular anethole, impart to an oral hygiene product according to the invention (having a content of menthol) a certain sweetness and a gentler, softer taste profile, and at the same time reduce the clove-like and medicinal taste impression of the eugenol acetate.

The weight ratio of eugenol acetate to anethole is advantageously in the range of 20:1 to 1:10, preferably in the range of 10:1 to 1:5.

The anethole can be used here as cis- or trans-anethole or in the form of mixtures of the isomers. Anethole can be employed here in the pure form (natural or synthetic) and/or as a constituent of natural oils and/or anethole-containing fractions of natural oils, in particular in the form of aniseed oil, star aniseed oil or fennel oil or anethole-containing fractions thereof.

It has moreover been found that an oral hygiene product according to the invention has a taste which is improved further if it comprises an aroma composition which also contains, in addition to menthol and optionally one or more substances having an aniseed note, eucalyptol (1,8-cineol).

Eucalyptol can be employed here in the pure form (natural or synthetic) and/or as a constituent of natural oils and/or eucalyptol-containing fractions of natural oils, for example in the form of bay (leaf) oil, but eucalyptus oils from Eucalyptus fruticetorum and/or Eucalyptus globulus and/or eucalyptol-containing fractions thereof are preferred.

The weight ratio of eugenol acetate to eucalyptol is advantageously in the range of 50:1 to 1:25, preferably in the range of 30:1 to 1:10.

Eucalyptol imparts to an oral hygiene product according to the invention (having a content of menthol) a fresh head note and slightly camphorous aspects, and at the same time reduces the clove-like and medicinal taste impression of eugenol acetate.

It has moreover been found that an oral hygiene product according to the invention has a taste which is improved further if it comprises an aroma composition which also contains, in addition to menthol and optionally one or more substances having an aniseed note and/or eucalyptol, at least one herbal component.

Suitable herbal components are, in particular, basil oil, camomile oil, nutmeg oil, in particular nutmeg blossom oil=mace oil) myrrh oil, oregano oil, parsley (seed) oil, rosemary oil, sage oil (clary sage, Dalmatian or Spanish sage oil), thyme oil, juniper oil (in particular juniper berry oil) or fractions thereof.

In addition, it is advantageous if the oral hygiene product according to the invention contains at least one further aroma substance from the following group: methone, isomenthone, isopulegol, menthyl acetate, isoeugenol methyl ether, limonene, pinene (optionally as a constituent of a Eucalyptus globulus oil), carvone (optionally as a constituent of a spearmint oil), methyl salicylate (optionally as a constituent of a wintergreen oil) and thymol (optionally as a constituent of a thyme or oregano oil).

In a preferred embodiment, an oral hygiene product according to the invention contains:

0.05 to 3 wt. % eugenol acetate and

0.05 to 10 wt. %, preferably 0.05 to 5 wt. % of an aroma composition which contains menthol and optionally one or more substances having an aniseed note and/or eucalyptol and/or one or more herbal components. The weight ratio of eugenol acetate to menthol here is preferably in the range of 10:1 to 1:10.

In this context, the weight data for eugenol acetate and the aroma composition in each case relate to the total weight of the finished oral hygiene product.

In a further preferred embodiment, an oral hygiene product according to the invention contains:

0.05 to 3 wt. % eugenol acetate and

0.05 to 10 wt. %, preferably 0.05 to 5 wt. % of an aroma composition which contains racemic menthol and/or I-menthol, anethole, eucalyptol and optionally one or more herbal components, the weight ratio of eugenol acetate to menthol being in the range of 10:1 to 1:10, preferably in the range of 5:1 to 1:5.

In this context, the weight data for eugenol acetate and the aroma composition in each case relate to the total weight of the finished oral hygiene product.

In the preferred embodiments of oral hygiene products according to the invention, the aroma composition preferably comprises:

5 to 80 wt. %, preferably 10 to 60 wt. % menthol;

1 to 25 wt. %, preferably 2 to 20 wt. % of one or more substances having an aniseed note, preferably anethole;

0.5 to 50 wt. %, preferably 1 to 30 wt. % eucalyptol; and optionally

0.01 to 25 wt. %, preferably 0.1 to 15 wt. % of one or more herbal components, in each case based on the total weight of the aroma composition.

The sum of menthol, substance(s) having an aniseed note, eucalyptol and optionally herbal component(s) in the aroma composition here is preferably greater than or equal to 40 wt. %, particularly preferably greater than or equal to 50 wt. %, and particularly preferably greater than or equal to 60 wt. %, based on the total weight of the aroma composition.

The oral hygiene products which are preferred according to the invention, containing eugenol acetate and an aroma composition comprising menthol, not only prevent or combat bad breath, but at the same time impart a fresh, pleasant taste and breath.

It is advantageous to buffer the oral hygiene products according to the invention. A pH range of from 3.5 to 10.0 is preferred.

Eugenol acetate can be incorporated without problems into the usual oral hygiene formulations for oral hygiene products. Preferred oral hygiene products are, for example, dental creams, toothpastes, dental gels, mouth washes, mouth rinses, liquids for gargling and mouth or throat sprays, as well as sucking pastilles, sucking tablets, bonbons, chewing gums, chewing bonbons and dental care chewing gums.

It is also possible and usually advantageous to combine eugenol acetate with other starting substances, for example with other antimicrobially active substances, aroma substances, flavouring substances and/or auxiliary substances.

The oral hygiene products according to the invention can contain auxiliary substances such as are conventionally used in such formulations, e.g. preservatives, abrasives, antibacterial agents, antiinflammatory agents, irritation-preventing agents, irritation-inhibiting agents, antimicrobial agents, antioxidants, astringents, antiseptic agents, antistatics, binders, buffers, carrier materials, chelating agents, cell stimulants, cleansing agents, care agents, surface-active substances, deodorizing agents, softeners, bactericides, emulsifiers, enzymes, essential oils, film-forming agents, fixatives, foam-forming agents, foam stabilizers, substances for preventing foaming, foam boosters, gelling agents, gel-forming agents, moisture-donating agents, moisturizing substances, moisture-retaining agents, bleaching agents, optically brightening agents, dirt-repellent agents, friction-reducing agents, lubricants, opacifying agents, covering agents, gloss agents, polymers, powders, proteins, abrading agents, silicones, skin soothing agents, skin cleansing agents, skin care agents, skin healing agents, cooling agents, skin cooling agents, warming agents, skin warming agents, stabilizers, suspending agents, thickeners, vitamins, oils, waxes, fats, phospholipids, saturated fatty acids, mono- or polyunsaturated fatty acids, α-hydroxy acids, polyhydroxy-fatty acids, liquefiers, dyestuffs, colour-protecting agents, pigments, aromas, flavouring substances, odoriferous substances or other conventional constituents of a cosmetic or oral hygiene formulation, such as alcohols, polyols, electrolytes, organic solvents, sweeteners, sugar substitutes, silicas, calcium carbonate, calcium hydrogen phosphate, aluminium oxide, fluorides, salts of zinc, tin, potassium, sodium and strontium, pyrophosphates, hydrogen peroxide and hydroxylapatite.

If the (oral hygiene) formulation is a solution or lotion, solvents which can be used are, for example: water or aqueous solutions, oils, such as triglycerides of capric or of caprylic acid or also alcohols, diols or polyols of low C number and ethers thereof, preferably ethanol, isopropanol, propylene glycol, glycerol or ethylene glycol. Mixtures of the abovementioned solvents can of course also be used.

Examples of flavouring substances or aromas which can be a constituent of an oral hygiene product according to the invention, in addition to eugenol acetate, are to be found e.g. in K. Bauer, D. Garbe, H. Surburg, Common Fragrance and Flavor Materials, 4th ed., Wiley-VCH, Weinheim 2001 or also in S. Arctander, Perfume and Flavor Chemicals, vol. I and II, Montclair, N.J., 1969, author and publisher.

Examples which may be mentioned of natural aromas which can be a constituent of an oral hygiene product according to the invention, in addition to eugenol acetate, are: peppermint oils, spearmint oils, Mentha arvensis oils, aniseed oils, clove oils, citrus oils, camphor oils, cinnamon oils, cinnamon bark oils, winter-green oils, eucalyptus oils, Eucalyptus citriodora oils, fennel oils, ginger oils, camomile oils, caraway oils, citronella oils, lime oils, orange oils, bergamot oils, grapefruit oils, mandarin oils, rose oils, geranium oils, sage oils, parsley seed oils, yarrow oils, star aniseed oils, basil oil, bitter almond oils, thyme oils, juniper berry oils, rosemary oils, angelica root oils, vanilla extracts, and fractions thereof or contents isolated therefrom.

Examples which may be mentioned of uniform aroma substances which can be a constituent of an oral hygiene product according to the invention, in addition to eugenol acetate, are: anethole, menthol, menthone, isomenthone, menthyl acetate, menthyl propionate, menthofuran, mint lactone, eucalyptol (1,8-cineol), limonene, eugenol, thymol, pinene, sabinene hydrate, 3-octanol, carvone, gamma-octalactone, gamma-nonalactone, germacrene D, viridiflorol, 1,3E,5Z-undecatriene, isopulegol, piperitone, 2-butanone, ethyl formate, 3-octyl acetate, isoamyl isovalerate, hexanol, hexanal, cis-3-hexenol, linalool, alpha-terpineol, cis- and trans- carvyl acetate, p-cymol, damascenone, damascones, rose oxide, fenchol, acetaldehyde diethyl acetal, 1-ethoxyethyl acetate, cis-4-heptenal, isobutyraldehyde, isovaleraldehyde, cisjasmone, methyl dihydrojasmonate, anisaldehyde, methyl salicylate, 2′-hydroxypropiophenone, menthyl methyl ether, myrtenyl acetate, 2-phenylethyl alcohol, 2-phenylethyl isobutyrate, 2-phenylethyl isovalerate, cinnamaldehyde, geraniol and nerol. In the case of chiral compounds, the aroma substances can be in the form of a racemate or in the form of an individual enantiomer or in the form of an enantiomerically enriched mixture.

Advantageous aromas or aroma substances which can be constituents of an oral hygiene product according to the invention, in addition to eugenol acetate, are, for example, anisole, basil oil, bitter almond oil, camphor oil, citronella oil, citrus oils, Eucalyptus citriodora oil, eucalyptus oil, camomile oil, spearmint oil, lime oil, mandarin oil, clove oil, orange oil, peppermint oil, sage oil, thyme oil, wintergreen oils, cinnamon oil, cinnamon bark oil, I-menthol, menthone, 1,8-cineol (eucalyptol), carvone, alpha-terpineol, methyl salicylate, 2′-hydroxypropiophenone and menthyl methyl ether.

Compounds having a physiological cooling effect (cooling substances) which can be a constituent of an oral hygiene product according to the invention, in addition to eugenol acetate, are, for example, I-menthol, menthone glycerol acetal, menthyl lactate, substituted menthyl-3-carboxylic acid amides (e.g. menthyl-3-carboxylic acid N-ethylamide), 2-isopropyl-N,2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, menthylhydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and tri-oxaalkanoates, 3-menthyl methoxyacetate, ilcilin and 1-menthyl methyl ether. I-Menthol, menthone glycerol acetal, menthyl lactate, menthyl-3-carboxylic acid N-ethylamide, 3-menthoxypropane-1,2-diol, 2-hydroxyethyl menthyl carbonate, 2-hydroxypropyl menthyl carbonate, monomenthyl succinate, menthyl 2-pyrrolidin-5-onecarboxylate and 1-menthyl methyl ether are preferred.

Oral hygiene products which contain at least one, particularly preferably at least two further cooling substances, in addition to I-menthol, are preferred according to the invention.

Components which cause a warming, sharp, tingling or prickling sensation on the skin or on the mucous membranes, in particular aroma substances having a heat-generating effect and/or sharp-tasting compounds (sharp substances), and which can be a constituent of an oral hygiene product according to the invention, in addition to eugenol acetate, are, for example, capsaicin, dihydrocapsaicin, gingerol, paradol, shogaol, piperine, paprika powder, chilli pepper powder, extracts from paprika, extracts from pepper; extracts from chilli pepper; extracts from ginger roots, extracts from Aframomum melgueta, extracts from Spilanthes acmella, extracts from Kaempferia galanga, extracts from Alpinia galanga, carboxylic acid N-vanillylamides, in particular nonanoic acid N-vanillylamide, 2-nonenoic acid amides, in particular 2-nonenoic acid N-isobutylamide and 2-nonenoic acid N-4-hydroxy-3-methoxyphenylamide, alkyl ethers of 4-hydroxy-3-methoxybenzyl alcohol, in particular 4-hydroxy-3-methoxybenzyl n-butyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, (4-hydroxy-3-methoxyphenyl)acetic acid amides, in particular (4-hydroxy-3-methoxyphenyl)acetic acid N-n-octylamide, nicotinaldehyde, methyl nicotinate, propyl nicotinate, 2-butoxyethyl nicotinate, benzyl nicotinate and 1-acetoxychavicol.

Further components which can be a constituent of an oral hygiene product according to the invention, in addition to eugenol acetate, are e.g. substances for improving oral hygiene, such as, for example, dental care and/or refreshing substances. The substances for improving oral hygiene include, for example, substances for combating or preventing plaque, tartar or caries, and those for combating or preventing bad breath. Reference may be made in this connection to U.S. Pat. No. 5,043,154. Examples which may be mentioned are Zn salts, such as Zn citrate and Zn fluoride, Sn salts, such as Sn fluorides, Cu salts, fluorides, e.g. amine fluorides, alkali metal fluorides, such as Na fluoride, alkaline earth metals fluorides and ammonium fluoride, phosphates, pyrophosphates, fluorophosphates, such as Na monofluorophosphate and Al mono- and Al difluorophosphate, alpha-ionones, geraniol, thymol, isomenthyl acetate, panthenol (provitamin B₅), xylitol, allantoin, niacinamide (vitamin B₃), tocopheryl acetate (vitamin E acetate) and poloxamer.

An oral hygiene product according to the invention can also contain one or more further antimicrobial active compounds for improving oral hygiene, in addition to eugenol acetate. These active compounds can be hydrophilic, amphoteric or hydrophobic in nature. Examples which may be mentioned are: Triclosan, chlorhexidine and salts, peroxides, phenols and salts thereof, domiphen bromide (phenododecinium bromide), bromochlorophene, Zn salts, chlorophylls, Cu salts, Cu gluconate, Cu-chlorophyll, sodium lauryl sulfate, quaternary monoammonium salts, such as coconut alkyl-benzyldimethylammonium chloride, or also pyridinium salts, such as cetylpyridinium chloride. In addition to individual active compounds, mixtures of active compounds or natural extracts or fractions thereof comprising active compounds can be employed, such as e.g. those obtainable from neem, berberitze, fennel, green tea, marigold, camomile, rosemary, thyme, propolis or turmeric.

An oral hygiene product according to the invention can contain antioxidants, in addition to eugenol acetate, and examples which may be mentioned here are: carotenoids, carotenes (e.g. α-carotene, β-carotene and lycopene) and derivatives thereof, flavonoids, quercetin (metal) chelators (e.g. α-hydroxy-fatty acids. fatty acids (palmic acids), phytic acid, lactoferrin, EDTA and EGTA), α-hydroxy acids (e.g. citric acid, lactic acid and malic acid), folic acid and derivatives thereof, ubiquinone and ubiquinol and derivatives thereof, vitamin C and derivatives (e.g. ascorbyl palmitate, Mg ascorbyl phosphate and ascorbyl acetate), tocopherols and derivatives (e.g. vitamin E acetate), vitamin A and derivatives (vitamin A palmitate), ferulic acid and derivatives thereof, butylhydroxytoluene (BHT), butylhydroxyanisole, zinc and derivatives thereof (e.g. ZnO and ZnSO₄), selenium and derivatives thereof (e.g. selenium-methionine); and constituents, extracts or fractions thereof isolated from plants, e.g. from tea, green tea, algae, grape seeds, wheat germ, camomile, rosemary and oregano.

An oral hygiene product according to the invention can contain dyestuffs, colouring agents or pigments, in addition to eugenol acetate, and examples which may be mentioned here are: lactoflavin (riboflavin), beta-carotene, riboflavin 5′-phosphate, alpha-carotene, gamma-carotene, cantaxanthin, erythrosine, curcumin, quinoline yellow, Yellow Orange S, tartrazine yellow, bixin, norbixin (annatto, orlean), capsanthin, capsorubin lycopene, beta-apo-8′-carotenal, beta-apo-8′-carotenic acid ethyl ester, xanthophylls (flavoxanthin, lutein, cryptoxanthin, rubixanthin, violaxanthin and rodoxanthin), fast carmine (carmic acid, cochineal), azorubine, Cochineal Red A (Ponceau 4 R), beetroot red, betanin, anthocyans, amaranth, Patent Blue V, Indigotine I (indigo carmine), chlorophylls, copper compounds of the chlorophylls, Brilliant Acid Green BS, lissamine green), Brilliant Black BN, carbo medicinalis vegetabilis, titanium dioxide, iron oxides and hydroxides, calcium carbonate, aluminium, silver, gold, Rubine Pigment BK (Lithol Rubine BK), Methyl Violet B, Victoria Blue R, Victoria Blue B, Acilan Brilliant Blue FFR (Brilliant Wool Blue FFR), Naphthol Green B, Acilan Fast Green 10 G (Alkali Fast Green 10 G), Ceres Yellow GRN, Sudan Blue II, ultramarine, phthalocyanine blue, phthalocyanine green and Fast Acid Violet R. Further naturally obtained extracts (e.g. paprika extract, black carrot extract, red cabbage extract) can be used for colouring purposes.

The following examples are intended to illustrate the present invention without limiting it.

### EXAMPLES

Unless stated otherwise, all the data relate to the weight.

### In Vitro Test for Reduction of Bad Breath

The test is based on the work of Goldberg and Rosenberg (Production of Oral Malodor in an in vitro System, S. Goldberg and M. Rosenberg, pp.143-150, in: Bad Breath—A Multidisciplinary Approach, eds: D. van Steenberghe, M. Rosenberg, Leuven University Press, 1996) and was adapted for better reproducibility.

A sterile liquid medium inoculated with fresh morning saliva is incubated at 37 ° C. for some days and then smelled by a panel of testers.

An intensive, typical bad breath odour had formed. Non-inoculated controls have only a weak smell of the medium.

Triclosan® (5-chloro-2-(2,4-dichlorophenoxy)phenol) was added to inoculated samples in a concentration of 0.05% as a control for the tests. After the incubation time, the inoculated and Triclosan®-treated samples had the same weak media smell as the non-inoculated samples.

By addition of 0.01% eugenol acetate to inoculated samples, no smell—analogously to Triclosan®—was to be found after the incubation time. This value also corresponds to the minimum active concentration. At significantly lower concentrations than 0.01%, a changed intrinsic smell or a mixed smell was perceptible in some cases.

If 0.05% eugenol acetate is added to the inoculated medium only one hour before the sensory evaluation, an unpleasant mixed smell of bad breath and a weak eugenol acetate note is to be observed. This observation demonstrates that the action of eugenol acetate against bad breath is not an odour-masking/-covering action.

### Determination of the Minimum Inhibitory Concentration

The minimum inhibitory concentration (MIC) of eugenol acetate was determined in the series dilution test against various bacteria. The result is shown in the following table:

| Organism | MIC [ppm] | Type |
|---|---|---|
| Escherichia coli | >1,000 | no action up to 1,000 ppm |
| Pseudomonas aeruginosa | >1,000 | no action up to 1,000 ppm |
| Staphylococcus aureus | >1,000 | no action up to 1,000 ppm |
| Streptococcus mutans | >1,000 | no action up to 1,000 ppm |
| Fusobacterium nucleatum | 500 | bactericidal |
| Prevotella intermedia | 500 | bactericidal |
| Porphyromonas gingivalis | 1,000 | |

A bactericidal action was thus detected against the germs Fusobacterium nucleatum and Prevotella intermedia, which cause bad breath, at a concentration of 500 ppm eugenol acetate. An activity against Porphyromonas gingivalis was furthermore observed, and an MIC of 1,000 ppm was determined.

In a further test in a concentration of 1,000 ppm, eugenol acetate showed no significant reduction in respect of the total number of germs in the oral saliva samples investigated.

### FORMULATION EXAMPLES

### 1. Gel Dental Cream with an Activity Against Bad Breath

| | I (%) | II (%) | III (%) |
|---|---|---|---|
| Na carboxymethylcellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na saccharinate | 0.07 | 0.07 | 0.07 |
| Na fluoride | 0.24 | 0.24 | 0.24 |
| p-Hydroxybenzoic acid (PHB) ethyl | 0.15 | 0.15 | 0.15 |
| ester | | | |
| Aroma A | 0.80 | 1.10 | 1.00 |
| Eugenol acetate | 0.025 | 0.06 | 0.2 |
| Abrasive silica | 11.00 | 11.00 | 11.00 |
| Thickening silica | 6.00 | 6.00 | 6.00 |
| Sodium dodecyl sulfate (SDS) | 1.40 | 1.40 | 1.40 |
| Dist. water | to 100.00 | to 100.00 | to 100.00 |

Aroma A here had the following composition:

30 wt. % I-menthol, 30 wt. % peppermint oil Mentha piperita, 21.5 wt. % peppermint oil Mentha arvensis, 9 wt. % anethole, 0.5 wt. % anisaldehyde, 2 wt. % eucalyptol, 1 wt. % Eucalyptus globulus oil, 3 wt. % menthone, 1 wt. % spearmint oil, 1 wt. % basil oil, 0.5 wt. % menthyl acetate, 0.05 wt. % menthyl lactate, 0.1 wt. % menthyl-3-carboxylic acid N-ethylamide (WS-3), 0.05 wt. % 2-hydroxyethyl menthyl carbonate (Frescolat MGC, Symrise), 0.05 wt. % 2-hydroxypropyl menthyl carbonate (Frescolat MPC, Symrise), 0.1 wt. % pinene, 0.1 wt. % propylene glycol, 0.05 wt. % limonene.

### 2. Dental Cream Against Plaque with an Activity Against Bad Breath

| | I (%) | II (%) | III (%) |
|---|---|---|---|
| Na carboxymethylcellulose | 1.00 | 1.00 | 1.00 |
| Glycerol | 12.50 | 12.50 | 12.50 |
| Sorbitol 70%, in water | 29.00 | 29.00 | 29.00 |
| Na saccharinate | 0.20 | 0.20 | 0.20 |
| Na fluoride | 0.22 | 0.22 | 0.22 |
| Azacycloheptane-2,2-diphospho acid, | 1.00 | 1.00 | 1.00 |
| di-sodium salt | | | |
| Bromochlorophene | 0.10 | 0.10 | 0.10 |
| Peppermint aroma | 1.10 | 1.10 | 1.10 |
| Eugenol acetate | 0.025 | 0.05 | 0.15 |
| Abrasive silica | 15.00 | 15.00 | 15.00 |
| Thickening silica | 5.00 | 5.00 | 5.00 |
| Sodium dodecyl sulfate (SDS) | 1.50 | 1.50 | 1.50 |
| Dist. water | to 100.00 | to 100.00 | to 100.00 |

### 3. Dental Cream Against Plaque with an Activity Against Bad Breath

Base: silica, alkali metal diphosphate

| | I (%) | II (%) | III (%) |
|---|---|---|---|
| Carrageenan | 0.90 | 0.90 | 0.90 |
| Glycerol | 15.00 | 15.00 | 15.00 |
| Sorbitol 70%, in water | 25.00 | 25.00 | 25.00 |
| PEG 1000 | 3.00 | 3.00 | 3.00 |
| Na fluoride | 0.24 | 0.24 | 0.24 |
| Tetrapotassium diphosphate | 4.50 | 4.50 | 4.50 |
| Tetrasodium diphosphate | 1.50 | 1.50 | 1.50 |
| Na saccharinate | 0.40 | 0.40 | 0.40 |
| Precipitated silica | 20.00 | 20.00 | 20.00 |
| Titanium dioxide | 1.00 | 1.00 | 1.00 |
| PHB methyl ester | 0.10 | 0.10 | 0.10 |
| Menthol/eucalyptol aroma | 1.10 | 1.10 | 1.10 |
| Eugenol acetate | 0.05 | 0.30 | 0.50 |
| Sodium dodecyl sulfate | 1.30 | 1.30 | 1.30 |
| Dist. water | to 100.00 | to 100.00 | to 100.00 |

### 4. Dental Cream Against Sensitive Teeth with an Activity Against Bad Breath

| | I (%) | II (%) | III (%) |
|---|---|---|---|
| Na carboxymethylcellulose | 0.70 | 0.70 | 0.70 |
| Xanthan gum | 0.50 | 0.50 | 0.50 |
| Glycerol | 15.00 | 15.00 | 15.00 |
| Sorbitol 70%, in water | 12.00 | 12.00 | 12.00 |
| K nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na saccharinate | 0.20 | 0.20 | 0.20 |
| Menthol/anethole aroma | 1.00 | 1.00 | 1.00 |
| Eugenol acetate | 0.025 | 0.10 | 0.20 |
| Ca carbonate | 35.00 | 35.00 | 35.00 |
| Silicon dioxide | 1.00 | 1.00 | 1.00 |
| Sodium dodecyl sulfate | 1.50 | 1.50 | 1.50 |
| (SDS) | | | |
| Dist. water | to 100.00 | to 100.00 | to 100.00 |

### 5. Dental Cream Against Sensitive Teeth with an Activity Against Bad Breath

| | I (%) | II (%) | III (%) |
|---|---|---|---|
| Hydroxyethylcellulose | 1.40 | 1.40 | 1.40 |
| Guar gum | 0.60 | 0.60 | 0.60 |
| Glycerol | 18.00 | 18.00 | 18.00 |
| Sorbitol 70%, in water | 12.00 | 12.00 | 12.00 |
| Na saccharinate | 0.35 | 0.35 | 0.35 |
| Dyestuff | 0.01 | 0.01 | 0.01 |
| PHB methyl ester | 0.15 | 0.15 | 0.15 |
| PHB propyl ester | 0.04 | 0.04 | 0.04 |
| Sr chloride | 10.50 | 10.50 | 10.50 |
| Peppermint/aniseed | 1.20 | 1.20 | 1.00 |
| aroma | | | |
| Eugenol acetate | 0.025 | 0.10 | 0.25 |
| Precipitated silica | 15.00 | 15.00 | 15.00 |
| Silicon dioxide | 1.60 | 1.60 | 1.60 |
| Sodium dodecyl | 1.30 | 1.30 | 1.30 |
| sulfate | | | |
| Dist. water | to 100.00 | to 100.00 | to 100.00 |

### 6. Ready-to-Use Mouth Wash with Fluoride and an Activity Against Bad Breath

| | I (%) | II (%) | III (%) |
|---|---|---|---|
| Ethanol | 7.00 | 7.00 | 7.00 |
| Glycerol | 12.00 | 12.00 | 12.00 |
| Na fluoride | 0.05 | 0.05 | 0.05 |
| Pluronic F-127 ® (BASF, | 1.40 | 1.40 | 1.40 |
| surface-active substance) | | | |
| Na phosphate buffer pH 7.0 | 1.10 | 1.10 | 1.10 |
| Sorbic acid | 0.20 | 0.20 | 0.20 |
| Na saccharinate | 0.10 | 0.10 | 0.10 |
| Menthol/peppermint aroma | 0.15 | 0.20 | 0.15 |
| Eugenol acetate | 0.01 | 0.05 | 0.08 |
| Dyestuff | 0.01 | 0.01 | 0.01 |
| Dist. water | to 100.00 | to 100.00 | to 100.00 |

### 7. Mouth Wash Concentrate with an Activity Against Bad Breath

| | I (%) | II (%) | III (%) |
|---|---|---|---|
| Ethanol, 95% strength | 80.00 | 80.00 | 80.00 |
| Na cyclamate | 0.15 | 0.15 | 0.15 |
| Menthol/aniseed/eucalyptol | 3.50 | 1.0 | 3.50 |
| aroma | | | |
| Dyestuff | 0.01 | 0.01 | 0.01 |
| Eugenol acetate | 0.50 | 1.5 | 2.5 |
| Dist. water | to 100.00 | to 100.00 | to 100.00 |

b 8. Chewing Gum Against Bad Breath

| | I (%) | II (%) | III (%) |
|---|---|---|---|
| Chewing gum base | 21.00 | 21.00 | 21.00 |
| Glucose syrup | 16.50 | 16.50 | 16.50 |
| Glycerol | 0.50 | 0.50 | 0.50 |
| Powdered sugar | 60.45 | 60.40 | 60.30 |
| Menthol/spearmint aroma | 1.50 | 1.50 | 1.20 |
| Eugenol acetate | 0.05 | 0.10 | 0.50 |

### 9. Sugar-Free Chewing Gum Against Bad Breath

| | I (%) | II (%) | III (%) |
|---|---|---|---|
| Chewing gum base | 30.00 | 30.00 | 30.00 |
| Sorbitol, powder | 38.45 | 38.40 | 38.30 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfame K | 0.10 | 0.10 | 0.10 |
| Emulgum/emulsifier | 0.30 | 0.30 | 0.30 |
| Sorbitol 70%, in water | 14.00 | 14.00 | 14.00 |
| Glycerol | 1.00 | 1.00 | 1.00 |
| Menthol/aniseed/cinnamon | 1.50 | 1.50 | 1.50 |
| aroma | | | |
| Eugenol acetate | 0.05 | 0.10 | 0.20 |

### 10. Gelatine Capsules Against Bad Breath for Direct Consumption

| | I (%) | II (%) | III (%) |
|---|---|---|---|
| Gelatine shell: | | | |
| Glycerol | 2.014 | 2.014 | 2.014 |
| Gelatine 240 Bloom | 7.91 | 7.91 | 7.91 |
| Sucralose | 0.065 | 0.065 | 0.065 |
| Allura Red | 0.006 | 0.006 | 0.006 |
| Brilliant Blue | 0.005 | 0.005 | 0.005 |
| Core composition: | | | |
| Plant oil triglyceride | 80.0 | 70.0 | 60.0 |
| (coconut oil fraction) | | | |
| Aroma B | 7.0 | 12.0 | 15.0 |
| Eugenol acetate | 3.0 | 8.0 | 15.0 |

Aroma B here had the following composition (data in each case in wt. %):

0.1% neotame powder, 0.05% aspartame, 29.3% peppermint oil arvensis, 29.3% peppermint piperita oil Willamette, 2.97% sucralose, 2.28% triacetin, 5.4% diethyl tartrate, 12.1% peppermint oil yakima, 0.7% ethanol, 3.36% 2- hydroxyethyl menthyl carbonate, 3.0% 2-hydroxypropyl menthyl carbonate, 0.27% vanillin, 5.5% D-limonene, 5.67% L-menthyl acetate.

The gelatine capsule, which is suitable for direct consumption, had a diameter of 5 mm, and the weight ratio of core material to shell material was 90:10. The capsules opened in the mouth within less than 10 seconds and dissolved completely within less than 50 seconds.

1. A method of inhibiting the growth of micro-organisms causing bad breath and/or to combat bad breath by adding thereto to a composition comprising eugenol acetate in an amount sufficient to act as an agent (i) for inhibiting and/or preventing the growth of and/or for killing microorganisms which cause bad breath and/or (ii) for combating bad breath.

2. A method according to claim 1, wherein the microorganisms which cause bad breath are chosen from the group consisting of: Eubacterium, Fusobacterium, Haemophilus, Neisseria, Porphyromonas, Prevotella, Treponema and Veillonella species.

3. An oral hygiene product containing eugenol acetate in an amount of at least 0.01 wt. %, based on the total weight of the product, wherein the weight ratio of eugenol acetate to eugenol is greater than 1:5 if the product contains eugenol.

4. An oral hygiene product according to claim 3, containing an amount of eugenol acetate in the range from 0.025 to 5.0 wt. %, based on the total weight of the product.

5. An oral hygiene product according to claim 3, wherein the weight ratio of eugenol acetate to eugenol is greater than 1.1.

6. An oral hygiene product according to claim 3, containing an aroma composition which comprises menthol and optionally one or more substances having an aniseed note and/or eucalyptol and/or one or more herbal components.

7. An oral hygiene product according to claim 3, containing
0.05 to 3 wt. % eugenol acetate and
0.05 to 10 wt. %, of an aroma composition which comprises menthol and optionally one or more substances having an aniseed note and/or eucalyptol and/or one of more herbal components.

8. An oral hygiene product according to claim 3, containing
0.05 to 3 wt. % eugenol acetate and
0.05 to 10 wt. %, of an aroma composition which comprises racemic menthol and/or 1-menthol, anethole, eucalyptol and optionally one or more herbal components, the weight ratio of eugenol acetate to menthol being in the range of 10:1 to 1:10.

9. An oral hygiene product according to claim 3, which is substantially free from eugenol.

10. A method of inhibiting and/or preventing the growth of and/or for killing microorganisms which cause bad breath comprising the following steps:
bringing microorganisms which cause bad breath into contact with an amount of eugenol acetate which is antimicrobially active against these microorganisms.

11. A method of combating bad breath comprising the following step
introducing an amount of eugenol acetate which is antimicrobally active against microorganisms which cause bad breath into the oral cavity and/or the pharyngeal cavity.
